(19) United States
(12) Patent Application Publication
(10) Pub. No.: US 2026/0020800 A1
(43) Pub. Date: Jan. 22, 2026

(54) INVASIVE DEVICE

(71) Applicant: AIRS MEDICAL INC., Seoul (KR)

(72) Inventor: Changkyun KIM, Gyeonggi-do (KR)

(21) Appl. No.: 19/132,877

(22) PCT Filed: Jun. 29, 2023
(86) PCT No.: PCT/KR2023/009100

§ 371 Date: May 27, 2025

(30) Foreign Application Priority Data
Dec. 2, 2022 (KR) 10-2022-0166763

(51) Int. Cl. A61B 5/15

(52) U.S. Cl. CPC A61B 5/15074; A61B 5/15003; A61B 5/150732

(57) ABSTRACT

An invasive device according to one embodiment of the present disclosure includes: a first guide part extended in a longitudinal direction; a first movement module guided along the first guide part; a second guide part extended in a longitudinal direction; and a second movement module guided along the second guide part; the first movement module is disposed in front of the second movement module; and the first and second movement modules are guided along the longitudinal directions, respectively.

### TECHNICAL FIELD

The present disclosure relates to an invasive device for automatically performing invasions of living bodies.

### BACKGROUND ART

In general, invasions of living bodies are performed for purposes such as blood collection, catheter sheath insertion, drug injection, etc.

Invasions of living bodies require precise punctures of blood vessels of invasion targets, so that it is important to locate blood vessels of the invasion targets. When blood vessels of invasion targets are not clearly visible, it is difficult to locate blood vessels, making invasions difficult.

To solve this problem, there was developed an invasive device that detected a blood vessel of an invasion target, automatically punctured the blood vessel of the invasion target with a needle, and then collected blood. Accordingly, this has brought about various changes in the health and medical field.

In addition, in order to perform invasions of living bodies for various purposes, it has become an important challenge to enable a single invasive device to perform multiple tasks.

### DISCLOSURE

### Technical Problem

The present invention has been conceived in response to the above-described background art, and an invasive device according to one embodiment of the present disclosure is intended to safely and accurately perform blood collection from a living body by performing a second operation mode.

An invasive device according to one embodiment of the present disclosure is intended to perform operations according to various invasive purposes through the individual operations of first and second movement modules based on preset operation modes by including the first and second movement modules in the invasive device.

An invasive device according to one embodiment of the present disclosure is intended to mount various types of invasive tools according to various invasive purposes by replacing and mounting first, second, and third adapters on first and second coupling parts.

However, the objects to be achieved in the present disclosure are not limited to the object mentioned above, and other objects not mentioned may be clearly understood based on the following description.

### Technical Solution

According to one embodiment of the present disclosure for achieving the above-described object, there is disclosed an invasive device. The invasive device according to the embodiment of the present disclosure includes: a first guide part extended in a longitudinal direction; a first movement module guided along the first guide part; a second guide part extended in a longitudinal direction; and a second movement module guided along the second guide part; the first movement module is disposed in front of the second movement module; and the first and second movement modules are guided along the longitudinal directions, respectively.

Alternatively, there may be provided the invasive device that may perform a first operation mode including an operation in which the first movement module is moved forward and the second movement module is moved rearward and a second operation mode including an operation in which the first movement module is moved forward and the second movement module is moved forward.

Alternatively, there may be provided the invasive device in which the first operation mode includes an operation in which the first and second movement modules complete primary forward movement and then the second movement module is moved rearward.

Alternatively, there may be provided the invasive device in which the first operation mode includes an operation in which the first movement module is moved forward secondarily and the second movement module is moved rearward.

Alternatively, there may be provided the invasive device in which the second operation mode includes an operation in which the second movement module is moved forward after the first movement module has completed forward movement.

Alternatively, there may be provided the invasive device in which the second operation mode includes an operation in which the second movement module is moved rearward after the second movement module has completed forward movement.

Alternatively, there may be provided the invasive device in which the invasive device includes a support part extended in the longitudinal direction and the support part is connected to the first and second movement modules and supports the guided movement of the first and second movement modules.

Alternatively, there may be provided the invasive device in which the first movement module includes a first coupling part for allowing a first adapter to be mounted thereinto and the first coupling part includes first fixation holes.

Alternatively, there may be provided the invasive device in which the first fixation holes are provided to be plural in number and are formed spaced apart from each other in the longitudinal direction.

Alternatively, there may be provided the invasive device in which the first movement module includes a first coupling part for allowing a first adapter to be mounted thereinto and the first coupling part includes a first magnetic portion.

Alternatively, there may be provided the invasive device in which the second movement module includes a second coupling part for allowing a second adapter to be mounted thereinto and the second coupling part includes second fixation holes.

Alternatively, there may be provided the invasive device in which the second fixation holes are provided to be plural in number and are formed spaced apart from each other in the longitudinal direction.

Alternatively, there may be provided the invasive device in which the second movement module includes a second coupling part for allowing a second adapter to be mounted thereinto and the second coupling part includes a second magnetic part.

Alternatively, there may be provided the invasive device in which the first adapter includes first fixation members for being inserted into the first fixation holes.

Alternatively, there may be provided the invasive device in which the first adapter includes a first magnetic body for being magnetically connected to the first magnetic part.

Alternatively, there may be provided the invasive device in which the second adapter includes second fixation members for being inserted into the second fixation holes.

Alternatively, there may be provided the invasive device in which the second adapter includes a second magnetic body for being magnetically connected to the second magnetic part.

Alternatively, there may be provided the invasive device in which the first movement module includes a third coupling part for allowing a third adapter to be mounted thereinto and the third adapter is intended for mounting a butterfly needle thereinto.

### Advantageous Effects

The invasive device according to one embodiment of the present disclosure has the effect of being able to safely and accurately perform blood collection from a living body by performing the second operation mode.

The invasive device according to one embodiment of the present disclosure has the effect of performing operations according to various invasive purposes through the individual operations of the first and second movement modules based on preset operation modes by including the first and second movement modules in the invasive device.

The invasive device according to one embodiment of the present disclosure has the effect of being able to mount various types of invasive tools according to various invasive purposes by replacing and mounting the first, second, and third adapters on the first and second coupling parts.

### DESCRIPTION OF DRAWINGS

FIG. 1 is a diagram showing an invasive device according to one embodiment of the present disclosure;

FIG. 2 is a diagram showing a coupling part and adapter of an invasive device according to one embodiment of the present disclosure;

FIG. 3 shows diagrams showing a catheter sheath and guide needle that may be mounted on adapters of an invasive device according to one embodiment of the present disclosure;

FIG. 4 shows diagrams illustrating the first operation mode of an invasive device according to one embodiment of the present disclosure;

FIG. 5 shows diagrams showing a blood collection needle and blood container that may be mounted on adapters of an invasive device according to one embodiment of the present disclosure;

FIG. 6 shows diagrams illustrating the second operation mode of an invasive device according to one embodiment of the present disclosure; and

FIG. 7 is a diagram showing a butterfly needle and an adapter according to one embodiment of the present disclosure.

### MODE FOR INVENTION

Embodiments of the present disclosure will be described in detail below with reference to the accompanying drawings so that those having ordinary skill in the art of the present disclosure (hereinafter, those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Additionally, in order to clearly describe the present disclosure, reference numerals for parts that are not related to the description of the present disclosure may be omitted in the drawings.

The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “at least one of A or B” used herein should be interpreted to refer to all combinations of A, B, and A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, the singular form should generally be construed to include “one or more.”

The term “N-th (N is a natural number)” used herein can be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

Meanwhile, the term “module” or “unit” used herein may be understood as a term referring to an independent functional unit processing computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of components of a computing device, an application program performing a specific function of software, a procedure implemented through the execution of software, a set of instructions for the execution of a program, or the like. Additionally, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The term “connected” used therein should be interpreted to include not only the case where components are “directly connected” to each other, but also the case where another component is “present” between components and the case where components are “electrically connected” with another component disposed therebetween.

The term “operation mode” used herein may mean a combination of operations that are performed according to a preset operation method and sequence in an invasive device. Furthermore, the “operation mode” may mean a combination of operations that are performed in response to a sensing value measured by a sensor according to a preset control method and sequence.

The foregoing descriptions of the terms are intended to help understanding of the present disclosure. Accordingly, it should be noted that unless the above-described terms are explicitly described as limiting the content of the present disclosure, the terms in the content of the present disclosure are not used in the sense of limiting the technical spirit of the present disclosure.

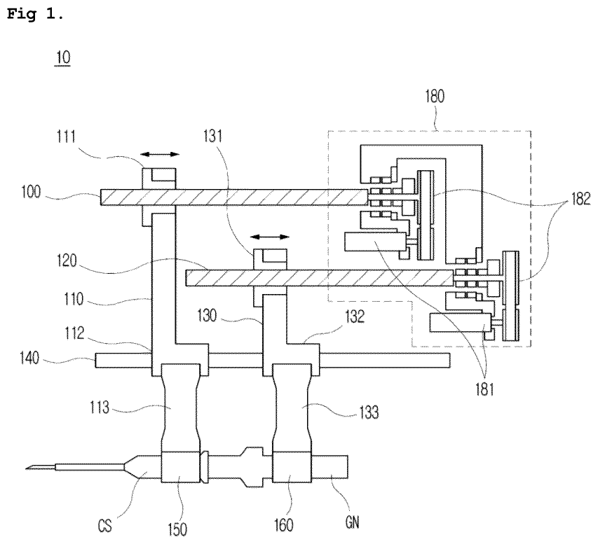

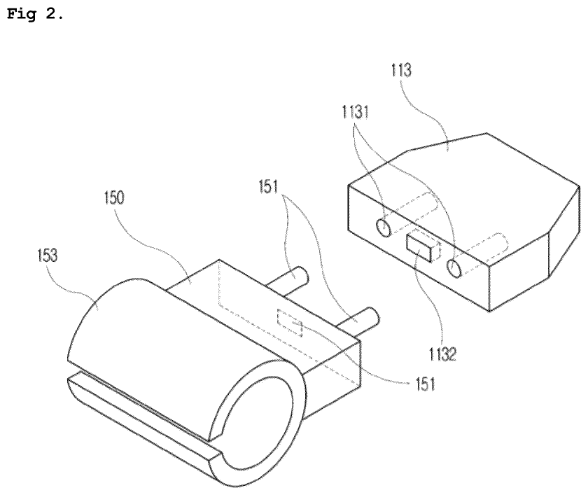

FIG. 1 is a diagram showing an invasive device according to one embodiment of the present disclosure. FIG. 2 is a diagram showing a coupling part and adapter of an invasive device according to one embodiment of the present disclosure.

Referring to FIGS. 1 and 2, an invasive device 10 according to one embodiment of the present disclosure may include a first guide part 100, a first movement module 110, a second guide part 120, a second movement module 130, a support part 140, a driving part 180, and a control unit (not shown).

The first guide part 100 may be extended in the longitudinal direction, and may be intended to guide the first movement module 110 through its movement. For example, the first guide part 100 may be a slide screw. The first guide part 100 may be configured to guide not only the slide screw but also the first slider 111 of the first movement module 110, to be described later, through their movement along the longitudinal direction.

The driving part 180 is connected to one end of the first guide part 100, and the first guide part 100 may be operated by the driving force provided by the driving part 180.

For example, the driving part 180 includes actuators 181 and gears and/or timing belts 182, and may be connected to the first guide part 100 and provide driving force to the second guide part 120. The driving part 180 may be configured to include not only the actuators 181 but also parts capable of providing power to the first guide part 100.

The first movement module 110 is guided along the first guide part 100, and may be mounted with a first adapter 150 to be described later. The first movement module 110 may include a first slider 111, a first guide hole 112, and a first coupling part 113.

The first slider 111 may be a part that is connected to the first guide part 100 described above and is moved in the longitudinal direction according to the operation of the first guide part 100. For example, the first slider 111 may be a slider that is moved in the longitudinal direction according to the rotation of the first guide part 100 having a slide screw shape. The first slider 111 may be provided at the top end of the first movement module 110.

The first guide hole 112 may be a part for being connected to the support part 140 to be described later. The first guide hole 112 may be a hole that penetrates in the longitudinal direction in the intermediate portion of the first movement module 110 extending downward from the first slider 111. The support part 140 may be inserted into the first guide hole 112.

The first coupling part 113 may be a part for allowing the first adapter 150, to be described later, to be mounted thereinto. The first coupling part 113 may include first fixation holes 1131 and/or a first magnetic part 1132.

The first fixation holes 1131 may be parts for allowing the first fixation members 151 of the first adapter 150, to be described later, to be inserted thereinto. The first fixation holes 1131 may be formed to be provided plural in number and spaced apart from each other in the longitudinal direction on the bottom surface of the first coupling part 113.

The first magnetic part 1132 may be a part for allowing the first magnetic body 152 of the first adapter 150, to be described later, to be magnetically attached thereto. The first magnetic part 1132 may be disposed in a portion between the first fixation holes 1131 provided spaced apart from each other on the bottom surface of the first coupling part 113. The first magnetic part 1132 may be disposed in the central portion of the bottom surface of the first adapter 150. The first magnetic part 1132 may be a magnet and/or a magnetic body.

The invasive device 10 according to one embodiment of the present disclosure may include the first adapter 150. The first adapter 150 may be intended for mounting a catheter sheath CS, or a blood collection needle IV. The first adapter 150 may include first fixation members 151, a first magnetic body 152, and a first mounting part 153.

The first fixation members 151 may be parts for being inserted into the first fixing holes 1131 described above. The first fixation members 151 may be formed to be plural in number and to protrude to be spaced apart from each other in the longitudinal direction on the top surface of the first adapter 150.

The first magnetic body 152 may be a part for performing magnetic attachment to the first magnetic part 1132 described above. The first magnetic body 152 may be disposed in a portion between the first fixation members 151 provided spaced apart from each other on the top surface of the first adapter 150. The first magnetic body 152 may be disposed in the central portion of the top surface of the first adapter 150. The first magnetic body 152 may be a magnet and/or a magnetic body.

The first mounting part 153 may be a part for allowing the catheter sheath CS or blood collection needle IV to be directly mounted thereinto. The first mounting part 153 may be provided in the form of a gripper and fix the position of the catheter sheath CS or blood collection needle IV by pressing the side of the catheter sheath CS or blood collection needle IV. Furthermore, the first mounting part 153 may be provided in the form of an elastic hole and fix the position of the catheter sheath CS or blood collection needle IV by pressing the side of the catheter sheath CS or blood collection needle IV.

The second guide part 120 may be extended in the longitudinal direction, and may be intended to guide the second movement module 130 through its movement. For example, the second guide part 120 may be a slide screw. The second guide part 120 may be configured to guide not only the slide screw but also the second slider 131 of the second movement module 130, to be described later, through their movement along the longitudinal direction. The second guide part 120 may be disposed below the first guide part 100.

The driving part 180 is connected to one end of the second guide part 120, and the second guide part 120 may be operated by the driving force provided by the driving part 180.

For example, the driving part 180 may include the actuators 181 and the gear and/or timing belts 182, and may be connected to the second guide part 120 and provide driving force to the second guide part 120. The driving part 180 may be configured to include not only the actuators 181 but also parts capable of providing power to the second guide part 120.

The second movement module 130 may be guided along the second guide part 120, and may be mounted with the second adapter 160 to be described later. The second movement module 130 may include a second slider 131, a second guide hole 132, and a second coupling part 133. The second movement module 130 may be disposed behind the second movement module 130.

The second slider 131 may be a part that is connected to the second guide part 120 described above and moved in the longitudinal direction according to the operation of the second guide part 120. For example, the second slider 131 may be a slider that is moved in the longitudinal direction according to the rotation of the second guide part 120 having a slide screw shape. The second slider 131 may be provided at the top end of the second movement module 130.

The second guide hole 132 may be a part for being connected to the support member 140 to be described later. The second guide hole 132 may be a hole that penetrates in the longitudinal direction in the intermediate portion of the second movement module 130 extending downward from the second slider 131. The support part 140 may be inserted into the second guide hole 132.

The second coupling part 133 may be a part for allowing the second adapter 160, to be described later, to be mounted thereinto. The second coupling part 133 may include second fixation holes (not shown) and/or a second magnetic part (not shown).

The second fixation holes may be parts for allowing the second fixing members (not shown) of the second adapter 160, to be described later, to be inserted thereinto. The second fixation holes may be formed to be provided plural in number and to be spaced apart from each other in the longitudinal direction on the bottom surface of the second coupling portion 133.

The second magnetic part may be a part for performing the magnetic attachment of the second magnetic body (not shown) of the second adapter 160 to be described later. The second magnetic part may be disposed in a portion between the second fixation holes provided spaced apart from each other on the bottom surface of the second coupling part 133. The second magnetic part may be disposed in the central portion of the bottom surface of the second adapter 160. The second magnetic part may be a magnet and/or a magnetic body.

The invasive device 10 according to one embodiment of the present disclosure may include the second adapter 160. The second adapter 160 may be intended for mounting a guide needle GN and a blood container VA. The second adapter 160 may include the second fixation members, a second magnetic body, and a second mounting portion (not shown).

The second fixation members may be part for being inserted into the second fixation holes described above. The second fixing member may be formed to be plural in number and to protrude to be spaced apart from each other in the longitudinal direction on the top surface of the second adapter 160.

The second magnetic body may be a part for performing magnetic attachment to the second magnetic part described above. The second magnetic body may be disposed in a portion between the plurality of second fixation members spaced apart from each other on the top surface of the second adapter 160. The second magnetic body may be disposed in the central portion of the top surface of the second adapter 160. The second magnetic body may be a magnet and/or a magnetic body.

The second mounting part may be a part for allowing the catheter sheath CS or blood collection needle IV to be directly mounted thereinto. The second mounting part may be provided in the form of a gripper and fix the position of the catheter sheath CS or blood collection needle IV by pressing the side of the catheter sheath CS or blood collection needle IV. Furthermore, the second mounting part may be provided in the form of an elastic hole and fix the position of the catheter sheath CS or blood collection needle IV by pressing the side of the catheter sheath CS or blood collection needle IV.

The support part 140 is connected to the first movement module 110 and the second movement module 130, and may be intended for supporting the guided movement of the first movement module 110 and the second movement module 130.

The support part 140 may be formed to extend in the longitudinal direction like the first guide part 100 and the second guide part 120. The support part 140 may have a shaft or bar shape. The support part 140 may be inserted and disposed in the first guide hole 112 and the second guide hole 132. The support part 140 may be disposed below the second guide part 120. The first guide part 100, the second guide part 120, and the support part 140 may extend in the longitudinal direction equally, and the central axes thereof may be disposed on the same vertical plane.

The control unit may be intended for controlling the operation of the driving part 180 according to the operation mode of the invasive device 10 to be described later. The control unit may include memory, a processor, and a communication module.

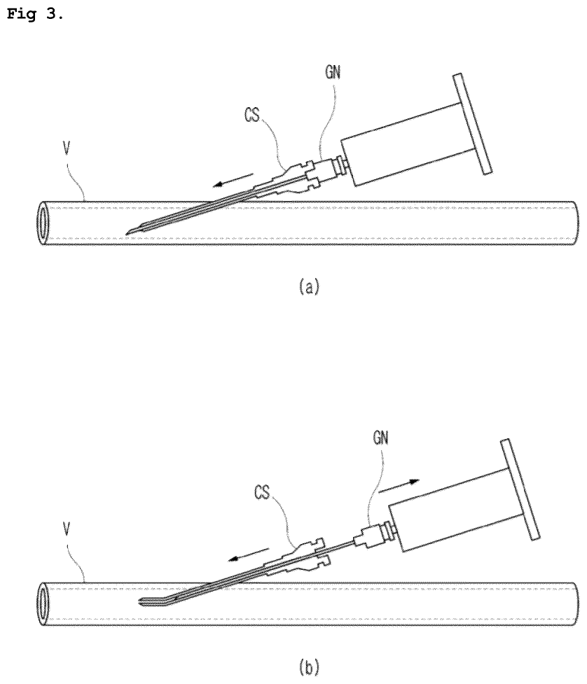

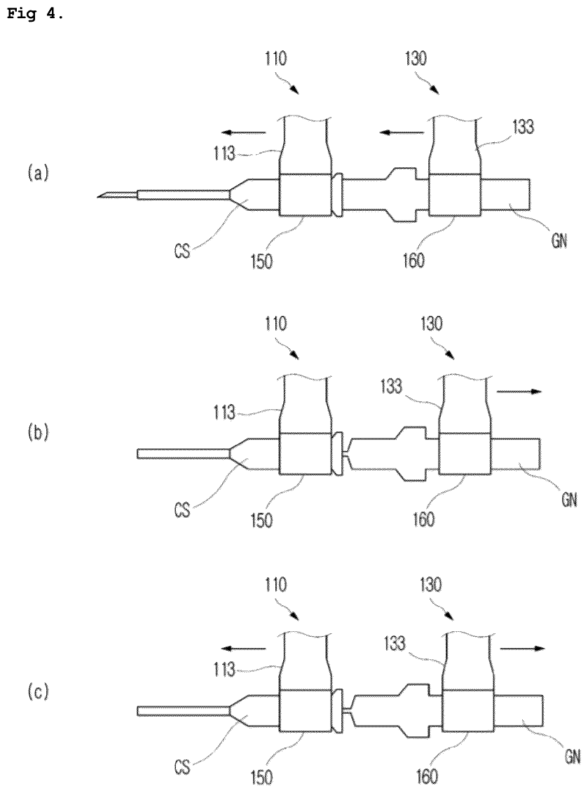

FIG. 3 shows diagrams showing a catheter sheath CS and guide needle GN that may be mounted on adapters of an invasive device 10 according to one embodiment of the present disclosure. FIG. 4 shows diagrams illustrating the first operation mode of an invasive device 10 according to one embodiment of the present disclosure.

Referring to FIGS. 3 and 4, the first operation mode may be an operation mode for inserting the catheter sheath CS into a living blood vessel V (see FIG. 3(a)) and inserting the catheter sheath CS deeper into the blood vessel V while ejecting the guide needle GN rearward (see FIG. 3(b)). The first movement module 110 may be mounted with the catheter sheath CS, and the second movement module 130 may be mounted with the guide needle GN.

The first operation mode may include an operation in which the first movement module 110 moves forward and the second movement module moves rearward.

More specifically, the first operation mode may first perform an operation in which both the first movement module 110 and the second movement module 130 move forward (see FIG. 4(a)). The first operation mode may perform an operation in which the first movement module 110 and the second movement module 130 complete primary forward movement and then the second movement module 130 moves rearward (see FIG. 4(b)).

Invasion may be performed while the first movement module 110 and the second movement module 130 are simultaneously moved forward primarily. When the invasion is completed, the first movement module 110 is stopped and the second movement module 130 is moved rearward, and thus, the guide needle GN may be ejected.

In addition, the first operation mode may perform an operation in which the first movement module 110 is moved forward secondarily and the second movement module 130 is moved rearward (see FIG. 4(c)). In the state in which the invasion is completed, the first movement module 110 is moved forward secondarily for additional insertion of the catheter sheath CS and the second movement module 130 is moved rearward, and thus, the guide needle GN may be ejected. When the additional insertion of the catheter sheath CS is completed, the secondary forward movement may be completed.

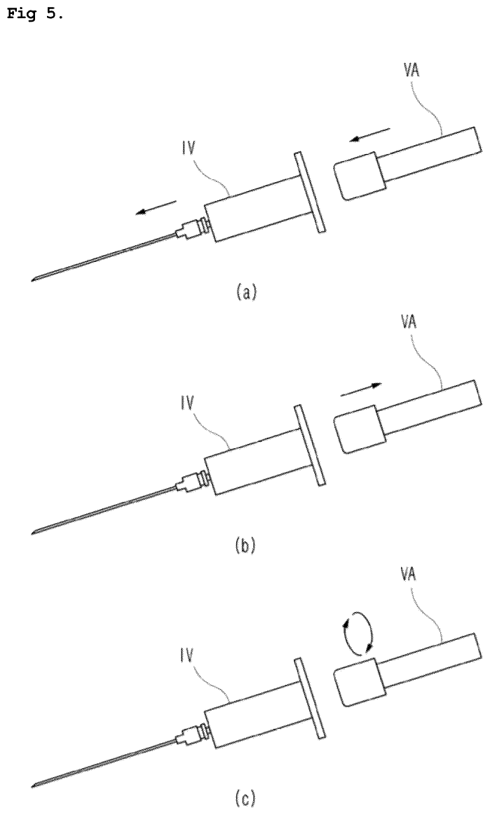

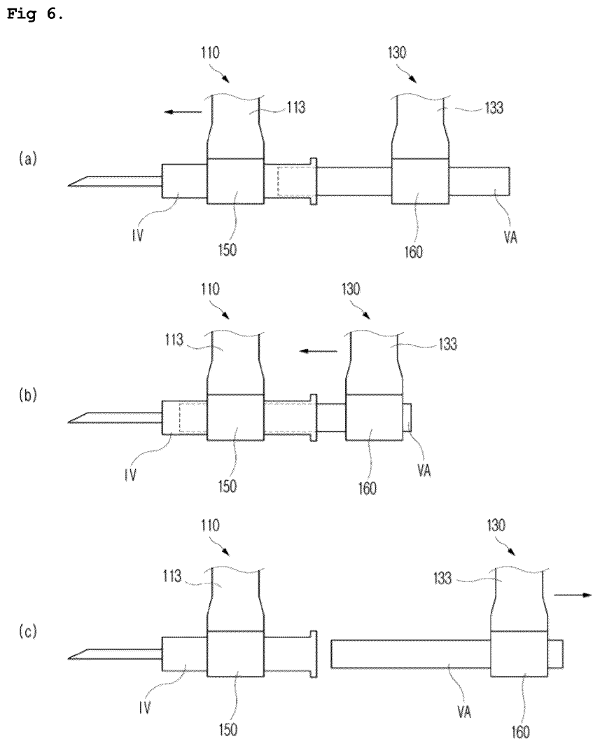

FIG. 5 shows diagrams showing a blood collection needle and blood container that may be mounted on adapters of an invasive device according to one embodiment of the present disclosure. FIG. 6 shows diagrams illustrating the second operation mode of an invasive device 10 according to one embodiment of the present disclosure.

Referring to FIGS. 5 and 6, the second operation mode may be an operation mode in which an operation of inserting the blood collection needle IV into a blood vessel for blood collection and connecting the blood collection needle IV and the blood container VA is performed (see FIG. 5(a)), an operation of, when blood collection is completed, moving the blood container VA rearward and thus separating it from the blood collection needle

IV is performed (see FIG. 5(b)), and finally an operation of moving the blood collection needle rearward is performed. The first movement module 110 may be mounted with the blood collection needle IV, and the second movement module 130 may be mounted with the blood container VA.

In addition, in the case where the blood collection needle IV performs invasion, blood collection may be repeated while the blood container VA is replaced and moved forward and rearward (see FIG. 5(c)).

The second operation mode may include an operation in which the first movement module 110 is moved forward and the second movement module 130 is moved forward.

More specifically, the second operation mode may perform an operation in which the first movement module 110 is moved forward (see FIG. 6(a)). Next, there may be performed an operation in which the second movement module 130 is moved forward (see FIG. 6(b)).

As the first movement module 110 is moved forward, invasion may be performed. In the state in which the forward movement of the first movement module 110 is completed and thus the invasion is completed, the second movement module 130 may be moved forward and thus the blood container VA may be connected to the blood collection needle IV, and blood collection may be performed by the difference in pressure between the blood pressure and the inside of the blood container VA.

The second operation mode may perform an operation in which the second movement module 130 is moved rearward after the second movement module 130 has completed forward movement (see FIG. 6(c)). The second movement module 130 may complete forward movement and stop, and then blood collection may be performed. After the blood collection has been performed, the second movement module 130 is moved rearward, and thus, the blood container VA may be separated from the blood collection needle IV.

In addition, in the state in which the first movement module 110 completes forward movement and invasion is completed, the second movement module 130 mounted with a first blood container VA may be moved forward to complete blood collection and be moved rearward, the blood container VA may be separated, the second movement module 130 may be mounted with a second blood container VA, be moved forward to complete blood collection and be moved rearward again, only the blood container VA may be replaced, and repeated blood collection may be performed.

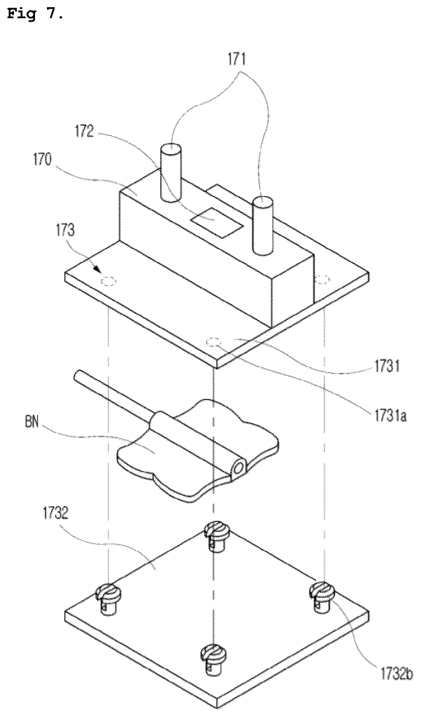

FIG. 7 is a diagram showing a butterfly needle and an adapter according to one embodiment of the present disclosure.

Referring to FIG. 7, the first movement module 110 may include a third coupling part for allowing a third adapter 170 to be mounted thereinto. The third coupling part (not shown) may be configured identically or similarly to the first coupling part 113 described above. The third coupling part may include third fixation holes (not shown) and a third magnetic part (not shown).

The third adapter 170 may be intended for allowing a butterfly needle BN to be mounted thereinto. The third adapter 170 may include third fixation members 171, a third magnetic body 172, and a third mounting part 173. The third fixation members 171 and the third magnetic body 172 may be configured identically or similarly to the first fixation members 151 and the first magnetic body 152 described above, respectively.

The third mounting part 173 may include an upper mounting part 1731 and a lower mounting part 1732. The upper mounting part and the lower mounting part 1732 may have plate shapes. Depressions 1731a may be formed on the upper mounting part 1731, and protrusions 1732a configured to be inserted into the depressions 1731a may be formed on the lower mounting part 1732. Alternatively, protrusions 1732a may be formed to protrude downward from the upper mounting part 1731, and depressions 1731a configure to receive the protrusions 1732a may be formed on the lower mounting part 1732. The butterfly needle BN may be disposed between the upper mounting part 1731 and the lower mounting part 1732, and may be pressed and fixed by the mutual coupling of the upper mounting part 1731 and the lower mounting part 1732.

The invasive device 10 according to one embodiment of the present disclosure described above has the effect of performing invasion so that the catheter sheath CS can be safely and accurately inserted into a living body by performing the first operation mode.

The invasive device 10 according to one embodiment of the present disclosure has the effect of being able to safely and accurately perform blood collection living body by performing the second operation mode.

The invasive device 10 according to one embodiment of the present disclosure has the effect of performing operations according to various invasive purposes through the individual operations of the first and second movement modules 110 and 130 based on preset operation modes by including the first and second movement modules 110 and 130 in the invasive device.

The invasive device 10 according to one embodiment of the present disclosure has the effect of being able to mount various types of invasive tools according to various invasive purposes by replacing and mounting the first, second, and third adapters 150, 160 and 170 on the first and second coupling parts 113 and 133.

The various embodiments of the present disclosure described above may be combined with one or more additional embodiments, and may be changed within the range understandable to those skilled in the art in light of the above detailed description. The embodiments of the present disclosure should be understood as illustrative but not restrictive in all respects. For example, individual components described as unitary may be implemented in a distributed manner, and similarly, the components described as distributed may also be implemented in a combined form. Accordingly, all changes or modifications derived from the meanings and scopes of the claims of the present disclosure and their equivalents should be construed as being included in the scope of the present disclosure.

1. An invasive device comprising:
a first guide part extended in a longitudinal direction;
a first movement module guided along the first guide part;
a second guide part extended in a longitudinal direction; and
a second movement module guided along the second guide part;
wherein the first movement module is disposed in front of the second movement module; and
wherein the first and second movement modules are guided along the longitudinal directions, respectively.

2. The invasive device of claim 1, wherein the invasive device:
performs a first operation mode including an operation in which the first movement module is moved forward and the second movement module is moved rearward; and
performs a second operation mode including an operation in which the first movement module is moved forward and the second movement module is moved forward.

3. The invasive device of claim 2, wherein the first operation mode includes an operation in which the first and second movement modules complete primary forward movement and then the second movement module is moved rearward.

4. The invasive device of claim 3, wherein the first operation mode includes an operation in which the first movement module is moved forward secondarily and the second movement module is moved rearward.

5. The invasive device of claim 2, wherein the second operation mode includes an operation in which the second movement module is moved forward after the first movement module has completed forward movement.

6. The invasive device of claim 5, wherein the second operation mode includes an operation in which the second movement module is moved rearward after the second movement module has completed forward movement.

7. The invasive device of claim 1, wherein:
the invasive device includes a support part extended in a longitudinal direction; and
the support part is connected to the first and second movement modules, and supports guided movement of the first and second movement modules.

8. The invasive device of claim 1, wherein:
the first movement module includes a first coupling part for allowing a first adapter to be mounted thereinto; and
the first coupling part includes first fixation holes.

9. The invasive device of claim 8, wherein the first fixation holes are provided to be plural in number, and are formed spaced apart from each other in a longitudinal direction.

10. The invasive device of claim 1, wherein:
the first movement module includes a first coupling part for allowing a first adapter to be mounted thereinto; and
the first coupling part includes a first magnetic portion.

11. The invasive device of claim 1, wherein:
the second movement module includes a second coupling part for allowing a second adapter to be mounted thereinto; and
the second coupling part includes second fixation holes.

12. The invasive device of claim 11, wherein the second fixation holes are provided to be plural in number, and are formed spaced apart from each other in a longitudinal direction.

13. The invasive device of claim 1, wherein:
the second movement module includes a second coupling part for allowing a second adapter to be mounted thereinto; and
the second coupling part includes a second magnetic part.

14. The invasive device of claim 8, wherein the first adapter includes first fixation members for being inserted into the first fixation holes.

15. The invasive device of claim 10, wherein the first adapter includes a first magnetic body for being magnetically connected to the first magnetic part.

16. The invasive device of claim 11, wherein the second adapter includes second fixation members for being inserted into the second fixation holes.

17. The invasive device of claim 13, wherein the second adapter includes a second magnetic body for being magnetically connected to the second magnetic part.

18. The invasive device of claim 1, wherein:
the first movement module includes a third coupling part for allowing a third adapter to be mounted thereinto; and
the third adapter is intended for mounting a butterfly needle thereinto.
